(19) United States
(12) Patent Application Publication
(10) Pub. No.: US 2022/0240833 A1
(43) Pub. Date: Aug. 4, 2022

(54) NEUROMONITORING DIAGNOSTIC SYSTEMS

(71) Applicant: Synchron Australia Pty Limited, Melbourne (AU)

(72) Inventor: Thomas James OXLEY, New York, NY (US)

(73) Assignee: Synchron Australia Pty Limited, Melbourne (AU)

(21) Appl. No.: 17/649,913

(22) Filed: Feb. 3, 2022

Related U.S. Application Data
(60) Provisional application No. 63/145,101, filed on Feb. 3, 2021.
(60) Provisional application No. 63/266,763, filed on Jan. 13, 2022.

(51) Int. Cl. A61B 5/37; A61B 5/00; A61B 5/375

(52) U.S. Cl. CPC A61B 5/37; A61B 5/0006; A61B 5/0031; G06F 3/015; A61B 5/4836; A61B 5/4848; A61B 5/375; A61B 5/6868

(57) ABSTRACT

Systems and methods to access the functionally distributed network of the brain to allow for directly accessing, monitoring, and/or communicating with specific regions of the brain when interacting with an external device.

### CROSS-REFERENCE TO RELATED APPLICATIONS

This application is a non-provisional of U.S. Provisional Application 63/145,101 filed on Feb. 3, 2021, and 63/266,763 filed on Jan. 13, 2022, the entirety of both of which are incorporated by reference.

### BACKGROUND

It is presently understood that in most cases a human brain functions as a well-coordinated network made up of collections of various brain regions, each an individual network of brain tissue and cells that is responsible for a specific purpose. Presently, statistical analysis of the functional magnetic resonance imaging (“fMRI”) allows neuroscientists to map the regions of the brain responsible for specific tasks. Furthermore, it is understood that many cognitive tasks are performed the networking of several discrete brain regions that are “functionally connected”. Therefore, the brain can be considered a distributed neural network that coordinates of a series of sub-networks associated with various regions of the brain, where each subnetwork is associated with a specific purpose.

Presently, conventional approaches exist that attempt to access these regions of the brain. Such approaches generally known include deep brain stimulation (“DBS”), which involve implanting electrodes within certain areas of a brain where the electrodes produce electrical impulses in an attempt to stimulate or regulate brain activity for a therapeutic or other purpose, as well as electrocorticography (“ECoG”), which enables neuromonitoring of brain regions for a diagnostic purpose.

DBS involves creating small holes in the skull to implant the electrodes and surgery to implant a controller or pacemaker-like that is electrically coupled to the electrodes to control the stimulation. Typically, this device is positioned under the skin in the chest. The amount of stimulation in deep brain stimulation can be controlled by the controller or pacemaker-like device where a wire/lead connects the controller device to electrodes positioned in the brain.

DBS can be used to treat a number of neurological conditions, such as tremors, Parkinson's disease, dystonia, epilepsy, Tourette syndrome, chronic pain, and obsessive-compulsive disorder. In addition, Deep brain stimulation has the potential for treatment of major depression, stroke recovery, addiction, and dementia. Moreover,

ECoG can provide a means of recording high-fidelity brain activity, for instance, during operations (intraoperative neuromonitoring) where real-time brain activity recordings may enable the treating surgeon to make immediate decisions that improves the safety of the treatment. Longer-term recordings are utilized for seizure detection in Epilepsy, and to assist in mapping to improve the safety of tumor resection by limiting removal of healthy brain. However, ECoG requires placement of electrode arrays directly onto the surface of the brain following exposure of the brain via craniotomy, for example, with subdural or epidural arrays. Therefore, their use is extremely limited in application.

FIG. 1 illustrates a conventional approach of accessing regions of the brain with a brain stimulation device 20 containing electrodes 22 that are implanted within a brain 12 of an individual 10. As shown, the implantation requires surgical penetration of the cranium 14 by the device 20 such that the device 20 is directed towards an area of interest 30. In addition, a lead 16 couples the device 20 to a controller/transceiver/generator 18.

There are a number of risks associated with the general surgery required to surgically implant the device 20 in conventional DBS procedures. Furthermore, there are risks in the process of the DBS procedure itself, given that conventional procedures require an approximation or non-invasive attempt to locate the region of interest 30. Then, the physician must attempt to physically position the electrodes 22 of the device 20 in or near the area of interest 30 such that the desired effect can be achieved. In certain cases, the positioning of the electrodes 20 can be a trial-and-error approach requiring multiple surgical attempts and multiple surgical insertion sites. Regardless of the number of attempts, the act of inserting the device 20 to position the electrodes 22 in the area of interest 30 creates collateral damage to brain tissue located in the path between the area of interest and the insertion point in the cranium.

Currently, the surgical risks involved in such procedures can include bleeding in the brain, stroke, infection, collateral damage to brain tissue, collateral damage to vascular structures in the brain, temporary pain, and inflammation at the surgical site. Apart from the surgical risks in conventional DBS involves risks in side effects of DBS if the electrodes stimulate or affect areas outside of the area of interest 30. Such risks can include breathing problems, nausea, heart problems, seizures, headache, confusion, etc. Yet an additional risk can be introduced upon attempting to remove a DBS device after a period of time given that tissue can heal around the device and implantation site.

However, the conventional approaches intended to access the many subnetworks of the brain are deficient such that the conventional approaches are unable to maximize the benefit of accessing and directly communicating/stimulating these subnetworks.

Conventional invasive approaches that involve direct brain penetration result in ongoing scar formation due to gliosis. Due to the nature of the level of invasiveness of craniotomy and the progressive nature of scar build up due to gliosis it is not feasible to remove and replace conventional DBS electrodes in the brain.

### SUMMARY

The ability to access the functionally distributed network of the brain allows for directly accessing, monitoring, and/or communicating with specific regions of the brain can allow for technological improvements in a number of areas, including but not limited to, healthcare, quality of life, improvements in the use of technology by an individual and improvements in the ability to communicate within a networked group of individuals. For example, direct access to this neural distributed network allows for improvement of conventional healthcare procedures for an individual and/or improvement of machine control by the individual. In an additional variation, the ability to directly access, monitor, and/or communicate with an individual's neural distributed network allows for improved communication with that individual and/or between individuals whose respective neural distributed networks are configured to directly network.

The present disclosure relates to systems and methods of facilitating direct interaction between a distributed neural network of a brain of an individual and an external device, the method including: generating a plurality of feedback data from the external device where the plurality of feedback data is related to an activity of the external device; establishing a connection from the external device to a control unit coupled to the individual, where the control unit includes at a first neural implant previously positioned within a first cytoarchitecture region of the distributed neural network of the brain of the individual; and transmitting the plurality of feedback data to the control unit, such that the control unit energizes the first neural implant to stimulate the first cytoarchitecture region of the brain, which produces an effect in the individual that is specific to the first cytoarchitecture region such that the individual is able to perceive the effect. Generating data from the external device can include data that is generated in/by the external device and/or data that is generated or measured separate/apart from the external device (e.g., via observation, tracking, etc.)

In some variations, the techniques described herein relate to a method wherein the plurality of feedback data is related to the activity of the external device resulting from actions of the individual.

Variations of the techniques described herein relate to a method wherein transmitting the plurality of feedback data occurs through a network. Alternatively, or in combination transmitting the plurality of feedback data occurs directly.

The methods and systems described herein can include variations where the individual is actively controlling and interacting with the external device during generating the plurality of feedback data.

Additional variations of the methods and systems include generating a plurality of outfeed data from a neural activity of the first cytoarchitecture region using the first neural implant and transmitting the plurality of outfeed data to the external device.

In some additional aspects, the techniques described herein relate to a method wherein transmitting the plurality of outfeed data to the external device includes transmitting at least one signal command representing the plurality of outfeed data.

The methods and techniques described herein can also include a second neural implant coupled to the control unit and positioned in a second cytoarchitecture region of the distributed neural network of the brain of the individual, and where transmitting the plurality of feedback data to the control unit includes energizing the first neural implant or the second neural implant, to stimulate the first cytoarchitecture region of the brain or the second cytoarchitecture region of the brain.

In some aspects, the techniques described herein relate to a method wherein the external device includes an external monitoring server and where the plurality of feedback data assists the individual in decision-making.

In additional aspects, the techniques described herein relate to a method wherein the external device includes a second control unit or a second electronic device, where the second control unit and the second electronic device are coupled to a second individual.

In additional variations, the external device includes a camera system worn by the individual.

An additional variation of systems and methods described herein includes facilitating direct interaction between a distributed neural network of a brain of an individual and an external device, the method including: generating a plurality of feedback data from the external device; establishing a connection from the external device to a control unit coupled to the individual, where the control unit includes at a first neural implant previously positioned within a first cytoarchitecture region of the distributed neural network of the brain of the individual; and transmitting the plurality of feedback data to the individual.

In some aspects, the techniques described herein relate to a method wherein transmitting the plurality of feedback data to the individual includes transmitting the plurality of feedback data to the control unit, such that the control unit energizes the first neural implant to stimulate the first cytoarchitecture region of the brain, which produces an effect in the individual specific to the first cytoarchitecture region such that the individual is able to perceive the effect.

Variations of the methods and systems include an external device comprising a position tracking system configured to monitor a position of the individual relative to an environment of the individual, wherein generating the plurality of feedback data from the external device includes information regarding an environmental condition around the individual.

Additional variations of the method and systems include transmitting the plurality of feedback data to an external hardware component which produces an effect perceivable by the individual. For example, the external hardware component can comprise a camera system worn by the individual.

An additional variation of the methods described herein include methods of assessing an effect of a medical procedure on a region of interest in a brain of an individual, the method including: positioning at least one endovascular neural monitoring implant within a vessel in the brain adjacent to the region of interest; engaging the individual to perform one or more tasks that induce a neural activity in the brain; and measuring the neural activity with the at least one endovascular neural monitoring implant to determine an association between the region of interest and the brain activity for assessing an effect of the medical procedure on the region of interest.

Variations of techniques described herein can relate to a method wherein positioning the at least one endovascular neural monitoring implant within the vessel in the brain adjacent to the region of interest includes positioning a plurality of endovascular neural monitoring implants within a plurality of vessels in the brain surrounding the region of interest.

The methods described herein can further include injecting a substance into the target area prior to measuring the neural activity. For example, the substance can include an anesthetic that is injected into an artery targeted for embolization. Moreover, the methods and systems can also further include measuring the neural activity with the at least one endovascular neural monitoring implant after injecting the substance.

In some aspects, the techniques described herein relate to a method, further including mapping the one or more tasks to one or more regions of the brain.

In some aspects, the techniques described herein relate to a method wherein measuring the neural activity further includes measuring the neural activity before and after the procedure.

Another variation of a method under the present disclosure includes methods of monitoring an epilepsy patient prone to seizures, the method including: positioning at least one endovascular neural monitoring implant within a vessel in the brain; monitoring a neural activity of the brain with the at least one endovascular neural monitoring implant over a period of time during which the individual ceases a seizure medication; and analyzing the neural activity to identify a region of the brain associated with a seizure.

In some aspects, the techniques described herein relate to a method wherein analyzing the neural activity to identify the region of the brain includes identifying the region of the brain active prior to the seizure.

In some aspects, the techniques described herein relate to a method of monitoring an individual in a clinically unresponsive state, the method including: positioning at least one endovascular neural monitoring implant within a vessel in the brain of the individual; providing an external stimuli to the individual while in the clinically unresponsive state; measuring a neural activity with the at least one endovascular neural monitoring implant during providing the external stimuli; assessing the neural activity to assess a condition of the individual.

In some aspects, the techniques described herein relate to a method, further including administering an anesthesia to the individual, measuring the neural activity after administering of the anesthesia and comparing the neural activity prior to and after administering of the anesthesia to obtain an indicator of brain function.

In some aspects, the techniques described herein relate to a method, further including delivering information regarding assessing the neural activity to a user interface of a caregiver,

In some aspects, the techniques described herein relate to a method wherein assessing the neural activity to assess the condition of the individual includes assessing the individual for information selected from the group consisting of: a prediction of outcome, a degree of recovery, and a measurement of improvement in the individual over time.

In some aspects, the techniques described herein relate to a method, further including comparing wherein assessing the neural activity to assess the condition of the individual using a dataset to predict recovery patterns of the individual.

### BRIEF DESCRIPTION OF THE DRAWINGS

FIG. 1 illustrates a conventional approach of accessing regions of the brain with a brain stimulation device containing electrodes that are implanted within a brain of an individual.

FIG. 2A shows an illustration of a cerebral cortex of a brain having a network of vasculature that supplies various regions of the cerebral cortex of the brain.

FIG. 2B is an illustration of the brain with the vasculature omitted to various cytoarchitecture regions of the cerebral cortex.

FIGS. 3A and 3B show variations of neural implants comprising an endovascular electrode array as part of a microwire monitoring/stimulation probe.

FIG. 4A illustrates a first variation of a system that directly accesses and monitors a specific region or subnetwork of a brain via a vascular approach.

FIG. 4B illustrates a variation of a system that directly accesses and monitors discrete regions or subnetworks of a brain via a vascular approach.

FIG. 4C illustrates a variation of a system that directly accesses and monitors discrete regions or subnetworks of a brain during a non-invasive medical procedure.

FIG. 5 illustrates a variation of a system comprising a plurality of neural implants comprising a number of microwire monitoring probes coupled to one or more monitoring devices.

FIG. 6 illustrates an application of the systems described herein using a distributed neural network of a brain for improved technology control, motor control, emotional monitoring, decision-making monitoring, sensory feed, auditory feed, visual feed, and communications to and from an individual.

FIG. 7 shows an example of a system using a distributed neural network of an individual for improved communication of data to and from the individual for improved interaction with any type of external device or machine.

FIG. 8 illustrates another variation of using a distributed neural network for improved communication of data to and from an individual for monitoring of the individual.

FIG. 9 illustrates a variation of using distributed neural networks to create a brain-to-brain network between at least two individuals.

FIGS. 10A and 10B show a first example of an individual having an implanted BCI vascular-based system.

FIG. 11A illustrates a variation of a system of the present disclosure having a camera system.

FIG. 11B illustrates a variation of a system of the present disclosure using a computing device to generate signals for neural stimulation.

FIG. 12 illustrates an example of an individual in an environment where a BCI stimulation apparatus receives input through any number of sensors or cameras to produce sensory stimulation to the individual.

### DETAILED DESCRIPTION

The present methods and devices relate electrodes that directly accessing, monitoring, and/or communicating with specific regions or subnetworks of the brain via a vascular approach for the purpose of using the direct access to send data to and out of the various subnetworks of a brain and associated nerves of an individual. As discussed below, the use of such data that is directly communicated to/from these neural subnetworks can improve any number of areas, including but not limited to medical applications, control of machines and electronic devices, real-time feedback on goal-oriented activity, as well as communication and consumer goods.

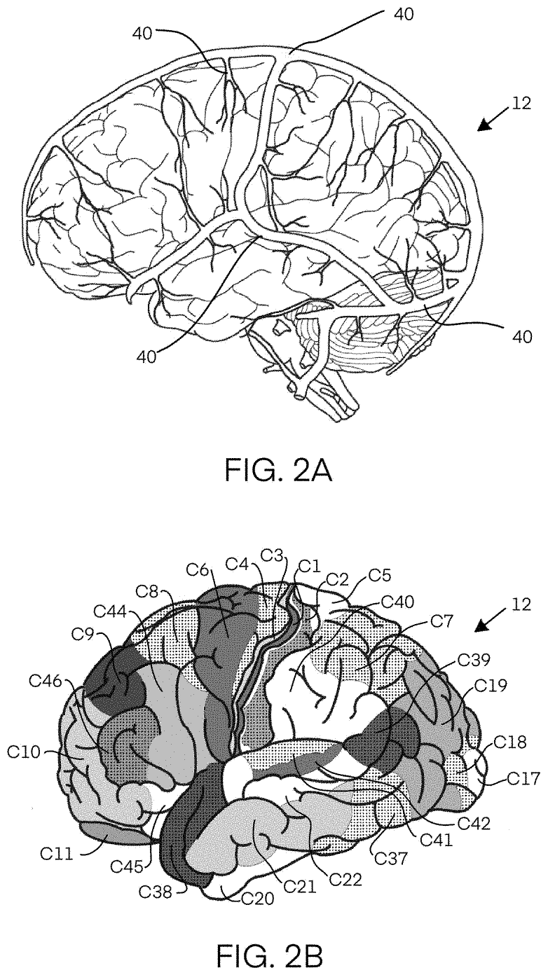

FIG. 2A shows an illustration of a cerebral cortex of a brain 12 having a network of vasculature 40 that supplies various regions of the cerebral cortex of the brain 12. The methods and devices described herein use the vasculature 40 to position one or more electrodes adjacent to a particular region of the brain. Variations of the methods can include using veins and/or arteries for positioning of the devices. In certain variations, the electrodes are positioned in veins to reduce inadvertently reducing or stopping blood flow to brain tissue. Moreover, as discussed below, the devices can be positioned entirely within a vessel. However, variations can include the use of devices or structures that penetrate the wall of a vessel.

FIG. 2B is an illustration of the brain 12 with the vasculature omitted to various cytoarchitecture regions (C1 to C46) of the cerebral cortex. These regions correspond to Brodmann areas that are based on an organization of neurons that were observed in the cerebral cortex that correspond to various cortical functions of the cerebral cortex. C1, C2, and C3 represent primary somatosensory cortex in the postcentral gyrus; C4 is the primary motor cortex; C5 is the superior parietal lobule; C6 is the premotor cortex and supplementary motor cortex; C7 is the visuo motor cortex; C8—includes frontal eye fields; C9—dorsolateral prefrontal cortex; C10—anterior prefrontal cortex (most rostral part of superior and middle frontal gyri); C11—orbitofrontal area (orbital and rectus gyri, plus part of the rostral part of the superior frontal gyrus); C17—primary visual cortex (V1); C18—secondary visual cortex (V2); C19—associative visual cortex (V3, V4, V5); C20—inferior temporal gyms; C21—middle temporal gyrus; C22—part of the superior temporal gyms, included in Wernicke's area; C37—fusiform gyrus; C38—temporopolar area (most rostral part of the superior and middle temporal gyri); C39—angular gyms, considered by some to be part of Wernicke's area; C40—supramarginal gyrus considered by some to be part of Wernicke's area; C41 and C42—auditory cortex; C44 and C45—Broca's area, includes the opercular part and triangular part of the inferior frontal gyrus; AND C46—dorsolateral prefrontal cortex.

The devices, methods, and systems described herein can benefit, or be combined with endovascular carriers and electrode arrays and systems/methods of using neural signals disclosed in the following patents and applications. U.S. Pat. No. 10,485,968 issued on Nov. 26, 2019, U.S. Pat. No. 10,512,555 issued on Dec. 24, 2019, U.S. Pat. No. 10,575,783 issued on Mar. 3, 2020, U.S. Pat. No. 10,729,530 issued on August 4, 2020, U.S. Pat. No. 11,093,038 issued on Aug. 17, 2021, and U.S. Pat. No. 11,141,584 issued on Oct. 12, 2021. U.S. Publication Nos.: US20210378595 published on Dec. 9, 2021, US20210393948 published on Dec. 23, 2021, US20200352697 published on Nov. 12, 2020, US20200078195 published on Mar. 12, 2020, US20190336748 published on Nov. 7, 2019, US20200016396 published on Jan. 16, 2020, US20210373665 published on Dec. 2, 2021, US20210342004 published on Nov. 4, 2021, US20210137542 published on May 13, 2021, US20210365117 published on Nov. 25, 2021, and US20210361950 published on Nov. 25, 2021. The contents of each of which are incorporated herein by reference in their entireties.

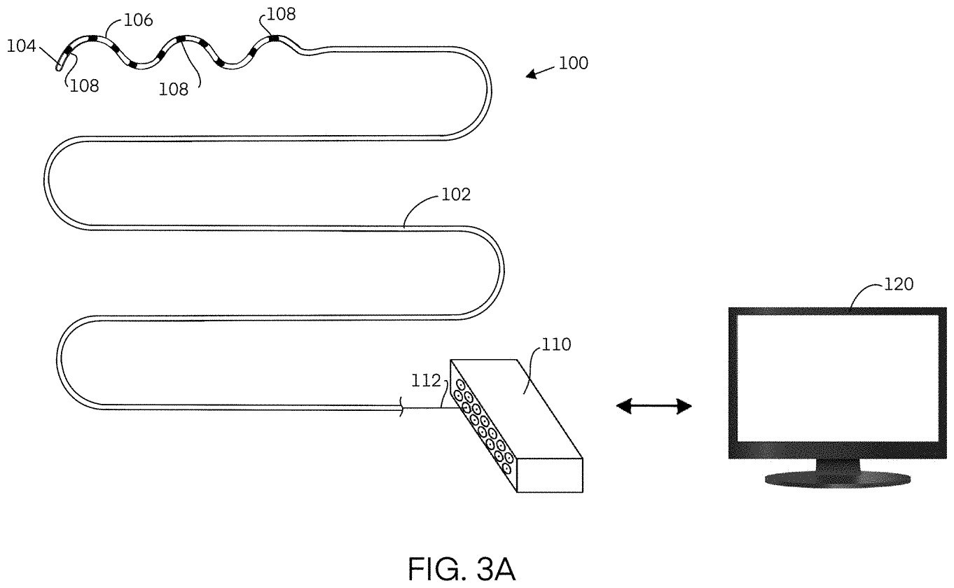

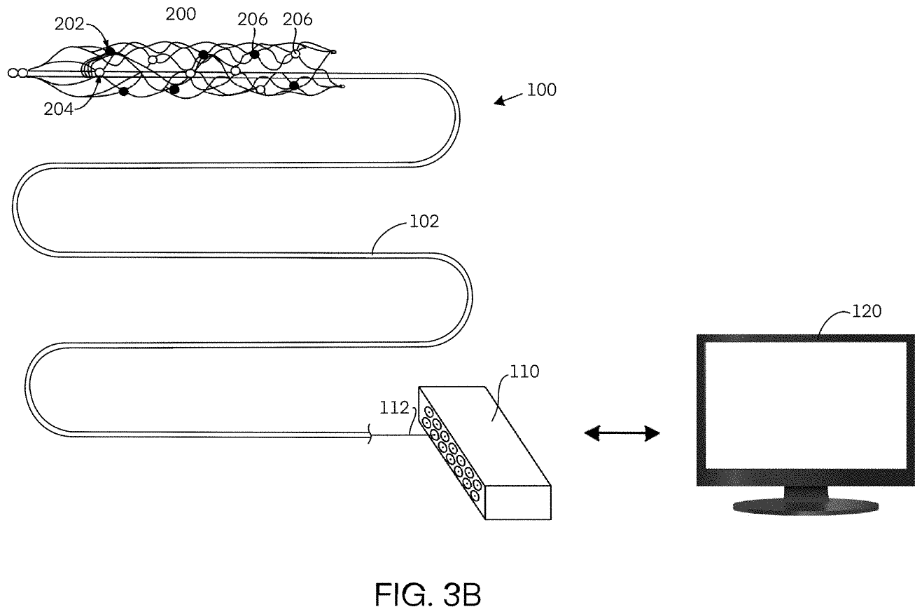

FIG. 3A shows an additional variation of a neural implant/neural implant 100 comprising an endovascular electrode array as part of a microwire monitoring/stimulation neural implant neural implant 100. As shown, the neural implant 100 includes one or more distal electrodes 108 located on a helical or sinusoidal 106 portion of a microwire 102. As discussed below, the non-linear distal portion 106 comprises an atraumatic tip 104 that allows for temporarily securing the electrodes 108 and distal portion 106 within a vessel within the brain without causing trauma to the vessel or brain. The non-linear shape 106 can function to provide apposition of the wire against the vessel wall as well position the electrodes 108 in contact with a vessel wall. The non-linear distal portion 106 can comprise a Nitinol material or core that allows the device to assume the non-linear shape when unrestrained or activated with current. The device can include one or more wires or cables configured to be wound or coiled. The wire or cable can be wound in a substantially helical pattern. In some embodiments, at least one of the first endovascular carrier and the second endovascular carrier can be a wire or cable comprising a sharp distal end for penetrating through lumen or vessel walls. Moreover, at least one of the first endovascular carrier and the second endovascular carrier can be a wire or cable comprising an anchor. For example, the anchor can be at least one of a barbed anchor and a radially-expandable anchor.

In additional variations, an entirety of the microwire 102 can comprise a shape memory alloy. In most variations, the neural implant 100 is configured to be removable from the vessel, for example, by pulling on the proximal end of the microwire 102. Additional variations of the device 100 include a non-linear shape at the electrode region 106 that can range from a helical shape, to a simple bend, or any shape that allows for anchoring in the delicate vessels of the brain. Alternatively, the series of electrodes 108 can be positioned on any structure that provides anchoring but does not restrict blood flow within the vessel.

The microwire 102 is typically sized in length and diameter such that it can be advanced into remote vasculature within the brain. For example, the diameter of the microwire 102 can range from can range from 0.010 to 0.018 inches. However, the size of the microwire should be chosen to allow advancement of the electrode portion into remote areas of the brain. Alternatively, proximal portion of the microwire 102 can have a larger diameter than the medial and distal regions to allow for increased pushability of the wire 102. The proximal end 112 of the microwire 102 is coupled to a connector base 110 that communicates using either a wireless or wired connection with monitoring software or other electronic/computing device 120.

In addition to being non-traumatic, variations of the monitoring probes/implants 100 described herein are configured to be removeable when used over a short time period. Alternatively, variations of monitoring probes can remain implanted over the span of months and/or years. In any case, the neural implant 100 can have anti-thrombotic coatings (e.g., heparin) to inhibit clotting of blood.

FIG. 3B illustrates another variation of a neural implant 100 comprising an endovascular carrier 200 (e.g., a stent structure) carrying different electrode arrays 202 and 204. Alternative variations can include a single electrode array or additional electrode arrays. As shown in FIG. 3B, two electrode arrays 202, 204 can be coupled to the same expandable structure 200 or endovascular scaffold. In other embodiments, three or more electrode arrays can be coupled to the same expandable stent or endovascular scaffold. Although FIG. 3B illustrates the electrodes 206 of the first electrode array 202 using dark circles and the electrodes 206 of the second electrode array 204 using white circles, it should be understood by one of ordinary skill in the art that the difference in color is only for ease of illustration.

Examples of additional neural implants can be found in U.S. Pat. Nos. 6,260,458; 6,428,489; 6,431,039; 6,440,088; 6,553,880; 6,579,246; and 6,766,720, the entirety of each of which is incorporated by reference. Additional neural implants

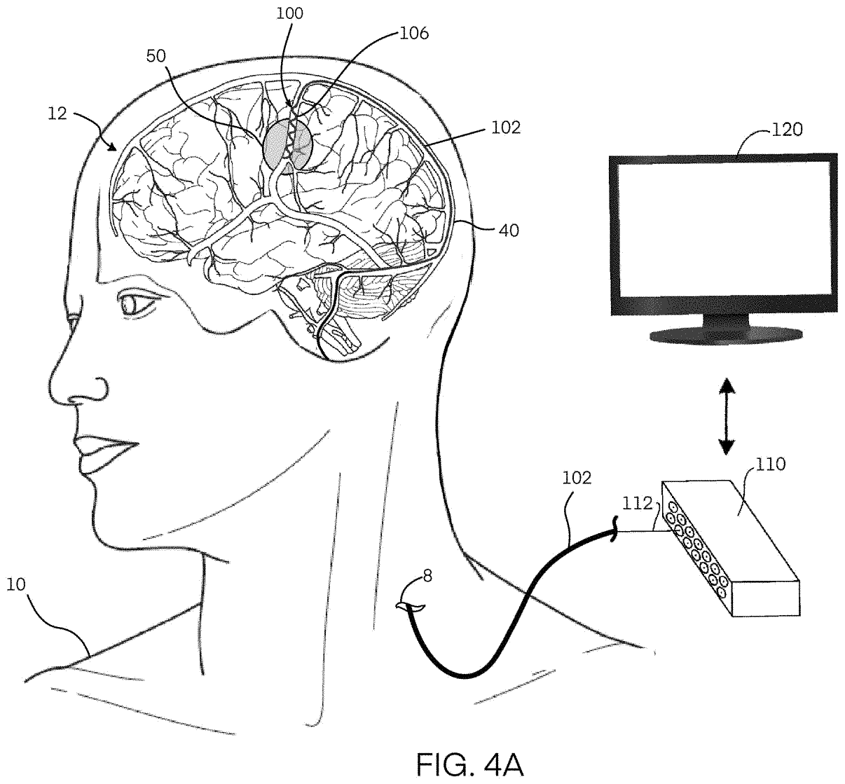

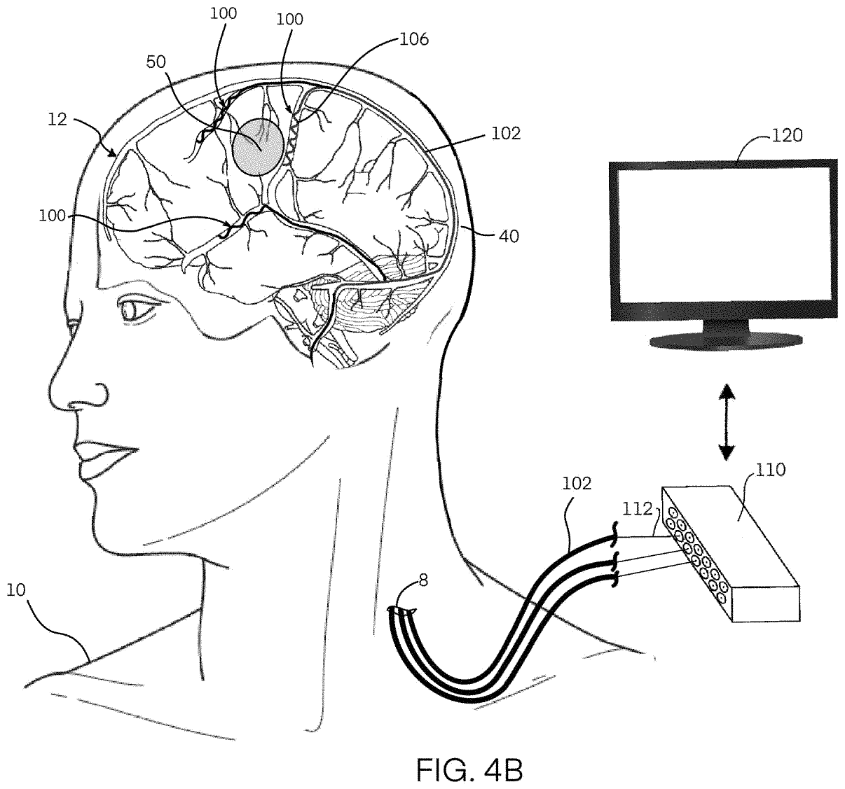

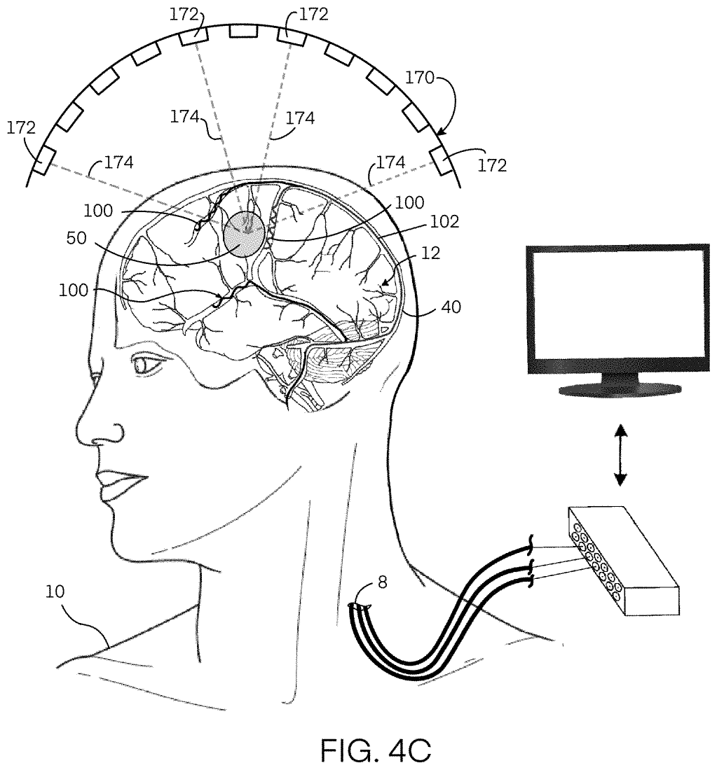

FIG. 4A illustrates a first variation of a system that directly accesses and monitors a specific region or subnetwork of a brain 12 via a vascular approach. In this variation, a microwire monitoring neural implant 100 is advanced through the vasculature into a vessel 40 within the brain 12. The illustrated variation is shown to be implanted for an operative procedure to provide brain monitoring during the procedure. In this variation, the neural implant 100 can be removed after the procedure or can remain implanted during a post-procedure monitoring period. Therefore, a proximal portion of a microwire 102 extends through one or more incisions 8 within the individual 10 for coupling to a controller 110 or another connector base. The system shown in FIG. 4A includes a single neural implant 100 for purposes of illustration. In practice any number of microwire monitoring probe devices can be used. Moreover, the neural implant 100 can be positioned using a microcatheter (not shown) that restrains the electrode portion 106 until deployed. Alternatively, a caregiver can directly advance the neural implant 100 in a linear configuration. Once positioned within a desired region, the caregiver can apply a current to the device to transform the electrode portion 106 from a linear configuration into the non-linear configuration so that the device remains anchored where desired.

As shown, a distal portion of the device 106 is configured to detect neural activity as well as remain temporarily anchored within a vessel. The neural implant 100 is deployed within a vessel and adjacent to a region of interest 50. In this example, the region of interest 50 represents an area of brain tissue that is intended to be removed or inactivated. Such procedures may involve tumor removal, removal of brain tissue to reduce epileptic seizures, treatment of arteriovenous malformations in the brain, etc. In conventional approaches, dye is used to identify the target region 50. Positioning one or more neural implants 100 in vessels adjacent to or surrounding the target region 50 allows for monitoring of neural signals at the site of the device deployment. The neural signals can be monitored before, during and after injection of the dye to see the effect of the dye or the procedure.

In additional variations, as shown in FIG. 4B, the devices described herein can replace or augment a Wada test that is performed on epilepsy patients considering surgery. In the Wada test, also known as the intracarotid sodium amobarbital procedure (ISAP), which establishes cerebral language and memory representation of each hemisphere. In the tests, which are conducted with the patient awake, a medical practitioner introduces a barbiturate (e.g., sodium amobarbital) into one of the internal carotid arteries via a cannula or intra-arterial catheter. Then the medical practitioner injects the drug into one hemisphere at a time into the right or left internal carotid artery to inhibit the respective side of the brain. For example, if the drug is injected into the right carotid, the right side of the brain becomes inhibited and cannot communicate with the left side of the brain. This allows the medical practitioner to observe the effect on any language and or memory function in that hemisphere in order to evaluate the other hemisphere. The test can also involve an EEG recording to confirm that the affected side of the brain is inactive. The practitioner can then engage the patient in language and memory-related tests. The present devices can allow for positioning of neural implants neural implants 100 within specific cytoarchitecture regions of the brain (see FIG. 2B) to record activity while the practitioner administers various memory, language, or psychological exercise that activates the brain. Detecting neural activity during the tests allows for mapping where that task is occurring from inside the brain. Once mapped, the practitioner can determine whether treatment/removal of the area of interest 50 can be performed and the potential consequences of doing so. In addition, the neural implants 100 can be used to monitor the various regions of the brain during the procedure as well as after the procedure.

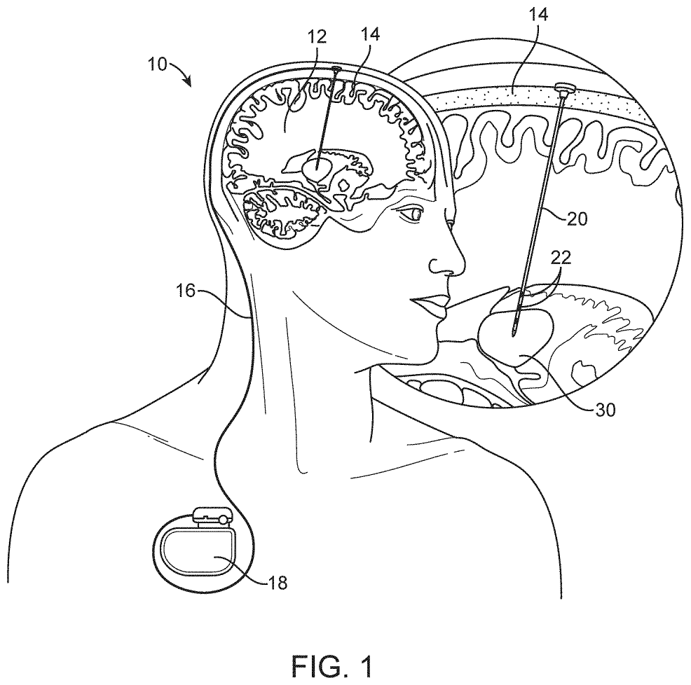

FIG. 4C illustrates an additional use of the methods and systems described herein for the non-invasive treatment of a region of interest 50. This region 50 can represent an area of brain tissue that is intended to be removed or inactivated. Such procedures may involve tumor removal, removal of brain tissue to reduce epileptic seizures, treatment of arteriovenous malformations in the brain, treatment of essential tremor, etc. As noted above, prior to treating tissue, one or more neural implants 100 are positioned in vessels, adjacent to, or surrounding the target region 50 allows for monitoring of neural signals at the site of the device deployment. Since the implants 100 are positioned using the vasculature, there is no need for any invasive penetration of the skull. However, variations of the method can include electrode implants, as shown in FIG. 1, to supplement the procedure. FIG. 4C illustrates an example of a focused ultrasound array 170 having a plurality of transducers 172 that deliver focused ultrasound energy 174 to the target region 50 to ablate the target region 50 or a portion of thereof. As discussed herein, the implants 100 and system can monitor neural activity before, during and/or after application of energy 174 to assess the effect of the treatment or to assess the collateral effect on brain activity as a result of the treatment.

In another variation, similiar to FIGS. 4A to 4C, the neural implants 100 may be placed in specific cytoarchitecture regions of the brain (see FIG. 2B) to aid in the safe intraarterial or intravenous embolization of arteriovenous malformations or malignancy. The use of neuromonitoring during endovascular embolization procedures involves the recording of evoked potentials from the brain using scalp-based EEG techniques. A somatosensory evoked potential is triggered by an electrical impulse delivered via electrical stimulation of the lower limb and recorded from the sensory cortex via EEG. To ensure the artery targeted for embolization is not supplying necessary brain function, an anesthetic agent such as lidocaine is injected into the artery. Any reduction in evoked potentials recorded during or immediately after the injection of lidocaine may be representative of potential brain injury that may occur, should an embolization agent be subsequently injected into said target artery. Intracranial endovascular ECoG recordings are of significantly higher sensitivity than scalp-based EEG and may represent an opportunity to improve the safety of intraoperative neuromonitoring during arterial embolization procedures.

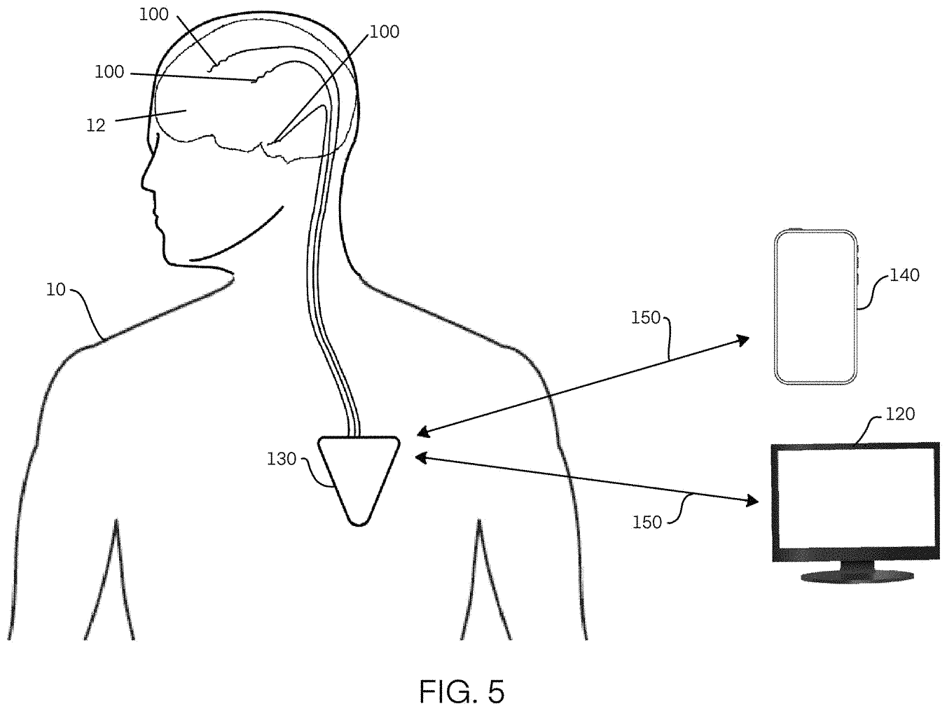

The systems described in FIGS. 4A to 4C are well suited for intraoperative and post-operative monitoring of the patient where there is little movement of the patient, such that the microwire(s) 120 can extend from one or more incisions 8 of the individual 10. FIG. 5 illustrates a variation of a system comprising a plurality of microwire monitoring neural implants 100 coupled to one or more monitoring devices 130. The monitoring device 130 can be fully, or partially implanted within the patient 10. Alternatively, the monitoring device 130 can be positioned outside of the body but allow for coupling with the neural implants 100 in a sterile manner. One of the purposes of the system shown in FIG. 5 is to provide in-patient or ambulatory monitoring of an individual 10. In such cases, the neural implants 100 remain within specific regions of the brain 12 for days or even months.

One application of the system shown in FIG. 5 involves monitoring of epilepsy patients, especially those patients that are not positively responding to medication. In such a case, any number of neural implants 100 are positioned within various regions of the brain 12. The neural implants 100 are coupled to a monitoring device that communicates 150 either via a wired or wireless connection to any number of electronic interface devices (e.g., a personal electronic device 140 or computer system 120). The patient 10 is then taken off seizure medication for a period of time, during which the system monitors brain activity through the neural implants 100. The activity is then analyzed to determine the regions of the brain that are associated with the seizures, including regions that are active prior to a seizure and/or regions that are responsible for the seizure. The implanted system allows for monitoring over days or even months. Current methods of determining brain areas associated with seizures involve craniotomy surgery, which removes part of the skull or cranium to access regions of the brain. The system shown in FIG. 5 performs brain-region-seizure mapping using a vascular approach. In additional variations, the systems described herein can be used in addition to conventional procedures.

The implanted unit 130 can include amplifiers, filters, controllers, data storage, a power supply, wireless communication equipment (e.g., RF, Bluetooth, etc.). Such equipment allows capturing of data over relatively long periods of time to provide the individual with mobility while being assessed.

In addition to brain mapping, by being implanted over a longer duration, the systems described herein can provide a warning system for patients that are subject to seizures. For example, the implants 100 can monitor various regions of the brain 12 and provide notifications via an external device (e.g., 140) or via the monitoring device 130 if the system detects that the individual is at a high risk of having a seizure. In such a case, the individual can be put on alert and avoid environments where a seizure would cause additional risk (e.g., driving, bathing, exercising, etc.). The system could also give varying levels of warning, such as low, medium, high risk of seizure that would allow an affected individual to have increased freedom from a sudden unexpected seizure.

In another variation, the systems described herein can also serve as a neuromonitoring diagnostic system that detects electrophysiological biomarkers in patients suffering from brain injury where the patient is otherwise unresponsive. Detection of the biomarkers can be an indicator of patient recovery. An example of such response is discussed in Classen, J. (2019). Detection of Brain Activation in Unresponsive Patients with Acute Brain Injury. The New England Journal of Medicine, 380(26) 2497-2505.

For example, in cases where a coma, stroke, hypoxic brain injury, or any brain injury renders the patient clinically unresponsive. The use of the systems described herein can assess the unresponsive patient for evidence of brain activation using ECoG in response to external stimuli, including auditory stimulus (e.g., spoken commands, familiar voices, etc.) and/or physical stimulus. In one variation, the purpose of the stimulus is to induce changes in brain state by interacting with the unresponsive patient. The neuromonitoring system can then provide a caregiver with a user interface/user exchange to provide various information to the caregiver regarding the condition of the patient. For example, the user interface can provide a prediction of outcome, degree of recovery, and/or measure improvement in the unresponsive patient over time. The measured response to the external stimulation can be compared to a dataset to predict recovery patterns of the patient. The dataset can be cloud based and updated based on machine learning algorithms that provide data standardization to provide a rating of the patient's condition, such as likely to improve or unlikely to improve.

The neuromonitoring system can also be combined with provocative testing where the patient is monitored in a resting state to determine activity and then again after an anesthesia is administered to the patient or a specific region of the patient's brain. The difference in the measured signals can be an indicator of brain function.

Use of the systems described herein as a neuromonitoring system allow for positioning of one or more endovascular electrode arrays in, for example, a motor region of the brain. However, the array(s) can be positioned in any number of regions of the brain. Implantation of the electrode array can be transitory where the array is removed after monitoring of the patient. Alternatively, the array can be implanted over a longer-term for increased monitoring of the patient. In either case, it may be desirable that the proximal end of the arrays is directly coupled to a controller/transceiver/generator that is not implanted in the patient (e.g., see FIG. 3).

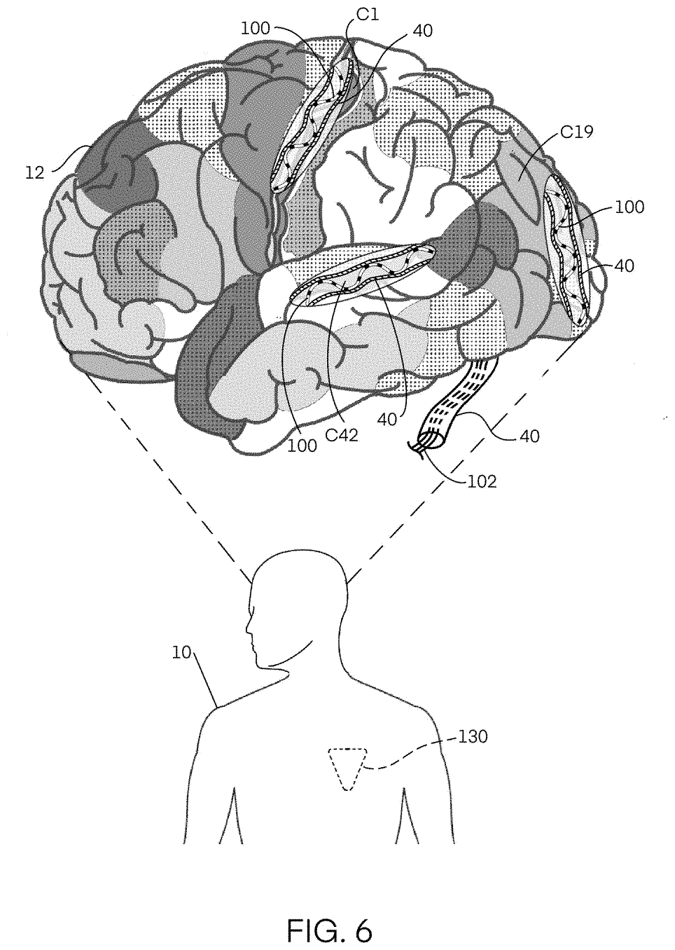

FIG. 6 illustrates another application of the systems described herein that use a distributed neural network of a brain for improved technology control, motor control, sensory feed, and communications to and from an individual. For example, FIG. 6 illustrates a magnified view of a brain 12 of an individual 10 having any number of microwire neural implants 100 positioned within vessels 40 associated with specific discrete cytoarchitecture regions (e.g., C1, C19, and C42) of the brain 12. Positioning the implants in discrete regions or networks of the brain allows sensory stimulation of or neural signal measurement in different regions of the brain to improve communication of data to and from the individual. In the illustrated example, the cytoarchitecture regions relate to auditory, sensory, and visual regions of the brain 12. However, these regions are chosen for illustrative purposes only. Additional variations of the systems and methods disclosed herein include any number of neural implants 100 positioned to be associated with any number of cytoarchitecture regions of the brain.

As shown in FIG. 6, the neural implants 100 are coupled to a control unit 130 via microwires 102 that extend through additional vasculature 40. As discussed below, the neural implants 100 allow data transfer to and from the various regions of the brain 12 to provide for improved communication of information to and from the individual 10 as well as improved control of electronic devices that are networked with the neural implants 100 and control unit 130. The regions illustrated in FIG. 6 are useful for data being in-fed to the individual. In additional variations, the regions of the brain useful for data being out-fed from the individual can comprise the same or different regions of the brain. For example, such regions can include areas of the brain responsible for language, decision prediction, motor control, emotions, etc.

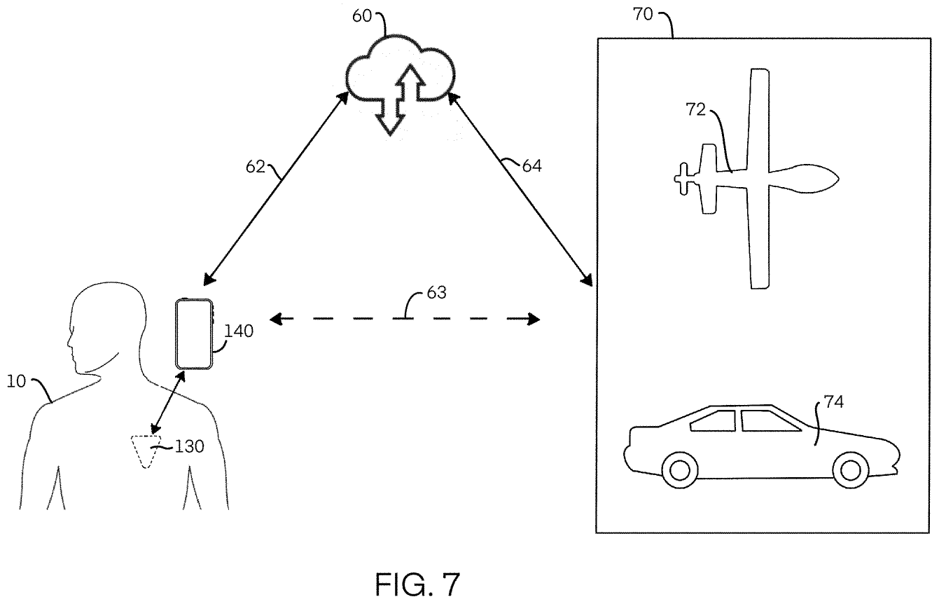

FIG. 7 shows one example of the systems described herein using a distributed neural network of an individual 10 for improved communication of data to and from the individual 10 for improved interaction with any type of external device or machine 70. FIG. 7 illustrates a vehicle such as a plane, drone 72, or automobile 74 for purposes of explaining the improved communication or data transfer. Clearly, the disclosure can include any machine or device configured for interaction with the individual 10.

In a conventional system, an operator controls a drone using a remote-control device along with an electronic interface that includes a screen providing various data of the operational parameters of the drone (e.g., speed, altitude, fuel, direction, etc.) The operator must observe these parameters in order to respond to any changing condition of the operational parameters. Next, the operator must formulate the thought for any subsequent action, and then to enact any corrective action. The operator must carry out the physical act of providing the drone with corrective action. While the operator might perform these actions quickly, there is a time delay between a change in condition of the drone, observing the change in condition, and then carrying out the physical corrective action to control the drone. Reaction speeds for vehicle operators require thoughts to be carried from their origin in the cortex through the spinal cord, peripheral nerves and ultimately to trigger muscle activity to enact the volitional command. Device (FIG. 5) enables information transfer at a speed superior to an unmodified human body.

In a system as shown in FIG. 7, data 64 concerning the drone's operating parameters can be transmitted to a network 62 or directly 63 to the electronics 140 that interface with a control unit 130 of the systems described herein either via a wired or wireless connection. (As discussed above, the use of a separate electronic unit 140 is optional for all the examples discussed herein.) As noted above (see FIG. 6), various probes can be positioned in different cytoarchitecture regions such that information 62 being transmitted to the individual can trigger stimulation of specific cytoarchitecture regions. For example, if the drone is gaining, altitude the system stimulates a first region of the brain, and if the drone is losing airspeed, the system can stimulate a second region of the brain. The operator will be trained to recognize the various stimulations to react accordingly. This direct transmission of data from a machine 70 to the system and individual 10 allows for a high-fidelity degree of control of the drone.

Additionally, the system can allow the individual 10 to use brain activity generated in a specific cytoarchitecture region to issue control commands to the drone. For example, if the individual 10 determines that the drone requires a course correction (e.g., move to the right), an implant positioned in a motor region of the individual will pick up brain activity of the individual who can produce a thought of a motor activity on their right side (e.g., pushing down with a right foot or activating a muscle on the right side). This neural activity is then transmitted via data 62, either through a network 60 or directly to the drone 72 such that the drone receives data 64 to automatically correct course. In both examples described herein, the system allows for direct communication between discrete regions of the brain and external machines 70 that require control. Alternatively, or in addition, transmitting data from the individual to the external device 70 can include transmitting a signal command that is determined by the neural activity of the individual 10. For example, if the system is configured so that when the individual produces a thought of a motor activity (as noted above) to provide a directional control to the external device 70, then the implant sensing the motor activity will generate outfeed data representative of that motor activity. Once the control unit 130 and/or electronics 140 identify that particular outfeed data, these components can issue a particular signal command to the external device that will be recognized by the external device (e.g., a directional command).

The system allows for improved control of the machines 70 as well as improved perception of the operating conditions of the machines. Although the above description discusses use of cytoarchitecture regions that control motor activity, any number of cytoarchitecture regions can be used, including but not limited to regions that control emotional broadcasting, language, decision prediction, visuospatial perception, auditory perception, and sensory perception (e.g., touch, smell, taste, etc.).

In yet a further variation, the system shown in FIG. 7 can use artificial intelligence or external data generated by a network 60 independently from the machine 70 or indirectly from the machine. For example, if an implant is positioned in a sensory region of the brain, triggering the implant can produce a perception of a smell, taste or similar sensory feed that is associated with a warning of some pre-determined condition. As one example, if an individual 10 is in a hostile territory and either the drone 72 or satellites have identified areas of actual or potential risk, the implant can be triggered to generate a specific perception that is associated with the area of actual or potential risk. The perception can be triggered to increase as individual moves toward the area and decrease when moving away. Alternatively, or in addition, this additional data can be used to feed an enemy's location through a visual cortex and represented through the brain of the individual 10 on to a Geo spatial representation to produce a direct visual feed into the brain from a control station.

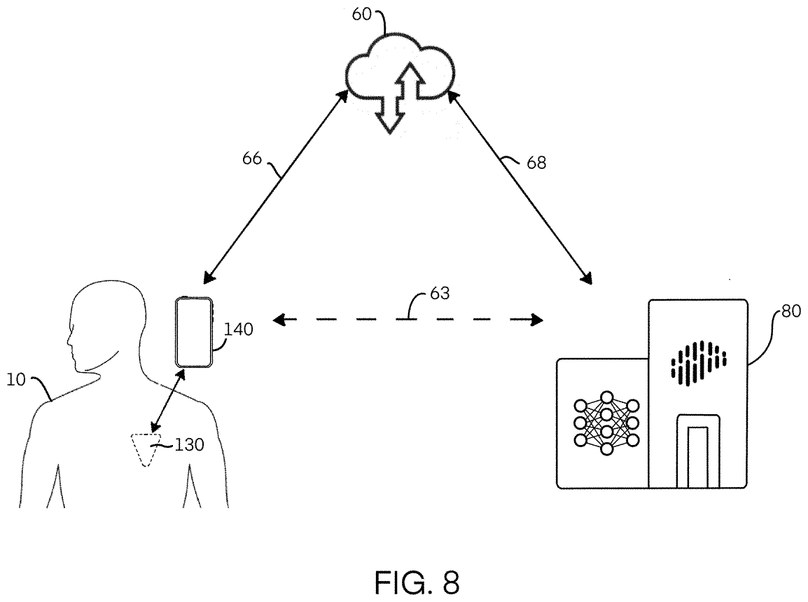

FIG. 8 illustrates another variation of using a distributed neural network for improved communication of data to and from an individual 10. In this variation, the implanted neural implant (shown as a microwire sensor) can be implanted a region of the brain corresponding to a prefrontal cortex, which is responsible for decision-making. Therefore, the implanted probe can generate signals that are predictive of decision-making. Such a feature can be used when the individual 10 is in a situation faced with making a difficult decision (e.g., a soldier, law enforcement, firefighter, etc.) The system can transmit data 66 68 to a monitoring site or server 80, which attempts to actually predict the way that the individual is making a decision and can then engage with the individual to assist, help, or even prevent the action. While FIG. 8 illustrates data transfer 66 and 68 occurring over a network 60, data transfer can also include direct data transfer to the control unit 130 and/or electronic device 140.

In a further variation, a tactical subject on a mission with limited communications to base command, such as an astronaut, utilizes the system for superior communication (e.g., with another astronaut or Mission Control). The device (FIG. 5) enables monitoring of the real-time cognitive activity of the astronaut, across the distributed cognitive domains (FIG. 2B) that aid in decision-making. For example, emotional arousal broadcasting, decision prediction and motor function can be monitored. Mission Control or additional individuals are further able to provide information to cognitive domains of the subject that can be received in various forms of perception, including sensory, auditory, visual and olfactory. For example, geospatial information to aid in decision-making during the mission can be provided directly into the visual cortex, and auditory feeds directly into the auditory cortex. The astronaut is then able to carry out the mission with a higher degree of precision by utilization of information flow directly into and out of cortex.

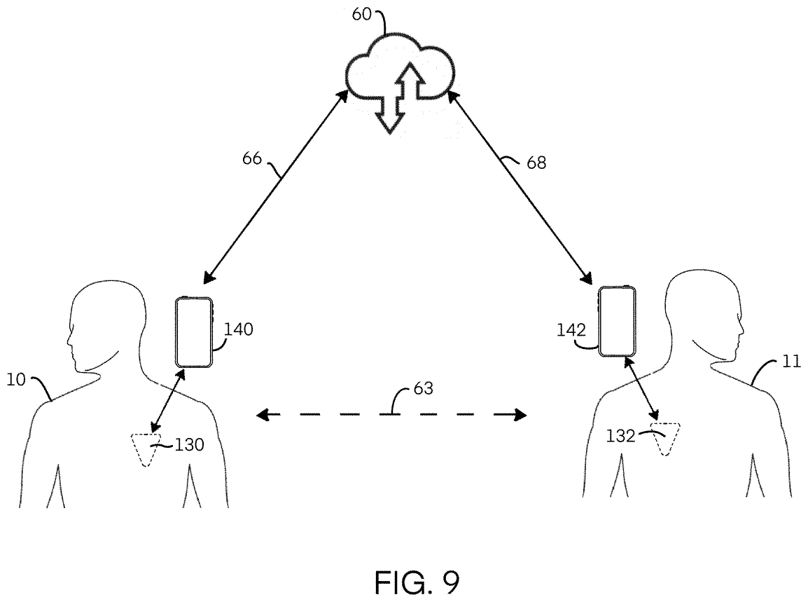

FIG. 9 illustrates another variation of using a distributed neural network by creating a brain-to-brain network between at least two individuals 10, 11 each having microwire monitoring/stimulation neural implants 100 respectively positioned in specific cytoarchitecture regions of their respective brain. FIG. 9 shows two individuals 10, 11 for purposes of illustration. However, the disclosure can include any number of individuals. As noted herein, the data transfer 66 and 68 between the individuals can rely on a network 60 or can occur directly through a local or private network. As also discussed, the system can include one or more electronic devices 140, 142 that communicate with a control unit 130, 132 that couples the probes or the electronic device 140, 142 can be respectively integrated into control units 130, 132. The example shown in FIG. 9 allows linking of two individuals 10, 11 through any number of means depending on placement of the devices in particular cytoarchitecture regions of the brain. In one example, the implants can be positioned in regions of the brain responsible for emotional responses so that each individual can be aware of an emotional component of the other. Such networking is not limited to emotions and can include connecting any region of the brain to provide sensory, motor, language, auditory, visual, taste, smell, etc. data communication directly between the individuals.

In a variation, a tactical cohort of subjects utilize networked brain function to achieve a superior level of information flow across the group. Being able to coordinate as one connected organism enables a superior group capacity to achieve a shared goal. In one example, a bright flare from an explosive may be viewed not only by a direct witness of the explosion, but by the entire group. An injury to one member of the group can be felt by the entire group. A shared consciousness across cognitive domains enables the group to perform at a higher function.

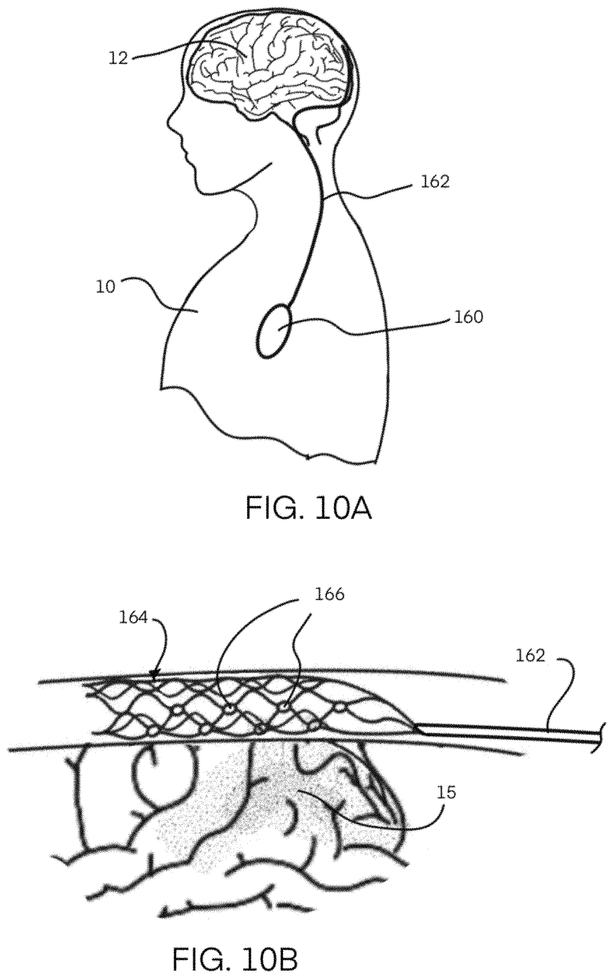

FIG. 10A illustrates an individual having a portion of a brain-computer-interface (BCI) as described in the patents and/patent applications listed above.

FIG. 10A shows a first example of an individual 10 having an implanted BCI vascular-base system. It is noted that variations of the methods described herein can apply to non-vascular-base BCI systems, including but not limited to direct placement of electrodes into brain/neural regions, external electrodes placed on the scalp or underneath an epidermal layer. FIG. 10A represents an individual 10 with a pulse generator 160 having a lead 162 or other electrical connection that delivers electrical signals to a stent positioned within the cerebral vasculature (the stent is not shown in FIG. 10A). In additional variations, the connection between the pulse generator 160 and stent can be wireless, which eliminates the need for a lead 102.

The stent is typically implanted proximate to various sensory tissues. In the present example, as shown in FIG. 10B, the stent 164 having electrodes 166 is positioned within a vessel 40 of the vasculature of the brain 12 such that it is adjacent to neural tissue 14 that can be stimulated to produce a recognizable effect in the individual. In one example, the implanted stent 164 can be implanted in sinus or venous tributaries that are adjacent to the visual (occipital) cortex. Additional areas include the venous sinuses: transverse sinus, straight sinus, super sagittal sinus; the superficial cortical veins: vein of labbe, inferior anastomotic vein; the deep venous cortical veins, basal vein of Rosenthal; and the posterior cerebral arteries. Moreover, variations of the method and systems can position a stent into a non-visual region of the brain as discussed below. Regardless, additional variation allow for placement of the stent 164 in any vessel that is adjacent to other regions of the brain.

As described in the patents and publications incorporated above, the stent 164 includes any number of electrodes that can be used to stimulate neural tissue 14 to produce a stimulation effect in the individual.

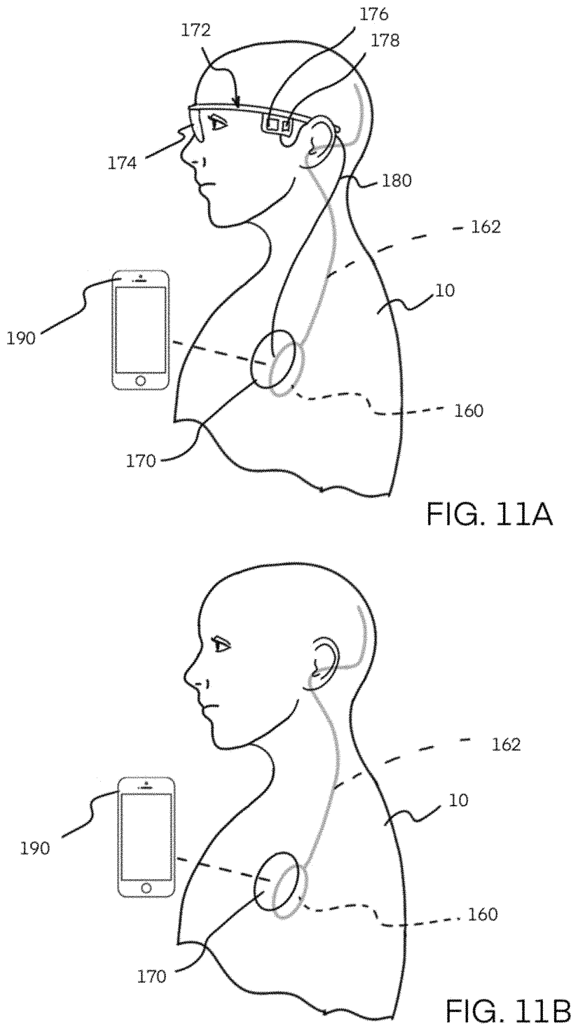

FIG. 11A illustrates a variation of a system of the present disclosure. As shown, an individual 10 can have a BCI system similar to that discussed above, where a pulse generator 160 is coupled by a lead 162 (or wirelessly) to a stent positioned within a brain tissue of the individual. The BCI can couple to an optional transmission unit 170 that relays signals to the pulse generator 160 for stimulation. Alternatively, any external hardware component can be configured to directly communicate (either wirelessly or wired) with the pulse generator.

In the illustrated example, a camera system 172 comprising a visual input/camera component 174 that communicates with a signal processor 176. The camera system 172 can include any number of power supplies 178 or other required electronics. During use, the camera system 172 is able to obtain information about the user's 10 surroundings and transmit image data to the signal processor 176, which in turn generates signals to cause the pulse generator 160 to ultimately stimulate a region of the individual's brain.

The camera system 172 can comprise any optical or other imaging system. For example, the camera system can use Lidar or other 3-dimensional imaging systems. The camera system 172 can include multiple cameras or lenses 174 as needed by the respective technology. Alternatively, or in combination, the camera system 172 can include an ultrasound-based system. Regardless of the imaging or sensing modality, the system generates input (either directly or a signal processor) so that for any given condition the pulse generator 160 stimulates a brain of the patient 2.

The systems described herein can provide stimulation sufficient to attempt to replicate vision in a patient. Alternatively, or in combination, the system can be configured to provide stimulation based upon certain environmental information. For example, if the camera system detects that the individual is approaching an object, the system can stimulate neural tissue in the individual's brain so that the individual perceives a sensory event. If the stimulation occurs in a visual (occipital) cortex the sensory event will be a visual event such as a flash or pulse that is perceived by the individual. In such a case, the sensory event can be a generic event (meaning that it must be associated) with any particular environmental sensory input. For example, the system can be configured such that if the camera system 160 identifies an object or obstruction at some distance from the individual, the system delivers a particular stimulation or sequence of stimulations that the individual would associate with the obstruction. In addition, the system can be configured to identify common environmental items such as stop lights, walk-signals, etc. and generate a sensory stimulation that the individual can associate with the respective environmental item. In each case, the sensory signal triggered in the individual would be ordinarily unassociated with the environmental item/obstacle but would be associated with the particular item/obstacle through configuration of the system.

The present system allows for any sensory stimulation that is triggered in the individual to be a universal stimulus that can be associated with any range of environmental conditions. Moreover, the combination and/or duration of the sensory stimulation can be further assigned to additional environmental conditions. In one basic example, the system can stimulate neural tissue so that an individual perceives a flash, where a rapid series of flashes can indicate that the individual should not proceed further, as in the case of the individual proceeding against a red-light or a do-not-walk signal. A slower series of flashes can be interpreted as a non-urgent cautionary.

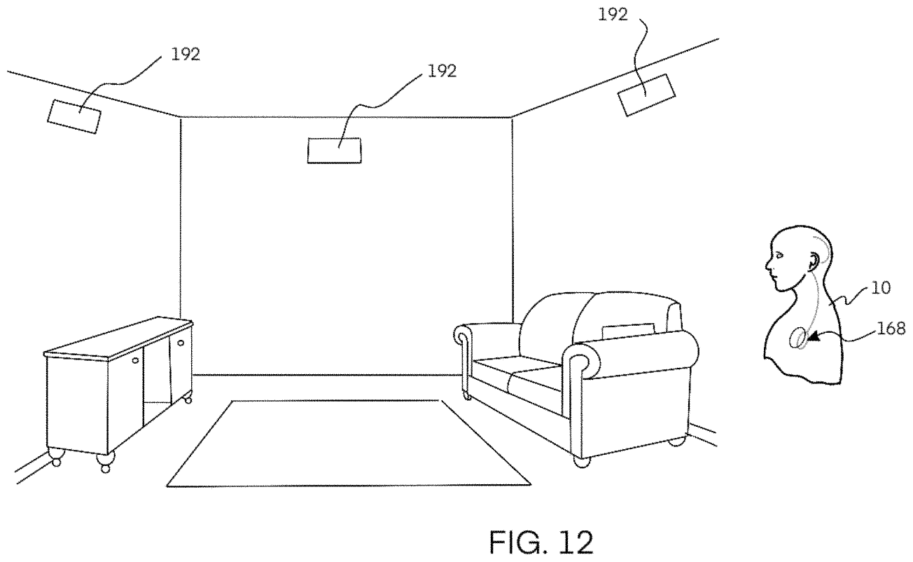

FIG. 11A and FIG. 11B both illustrate a computing device 190, which can include a smart-phone, tablet, or other computer that is also configured to interact with the pulse generator 160 either directly or using an intermediate component 118. Where the intermediate component 170 can optionally be coupled to the camera system 172 via a wireless or wired 180 connection. In either case, the system contemplates the use of a computing device 190 to with a camera system 172 (as shown in FIG. 12A) or without a camera system (as shown in FIG. 11B). In the variation where the BCI is used without a camera system, the computing device 190 can provide environmental information to the pulse generator 160 (which ultimately provides stimulation to the individual 2) through the use of GPS positioning information or through the use of an on-board camera on the computing device 190.

FIG. 12 illustrates an example of an individual 10 in an environment where the BCI stimulation apparatus 168 receives input through any number of sensors, cameras, or position tracking system 192 to produce sensory stimulation to the individual 2. In the illustrated example, the sensors/cameras 192 can provide environmental information as well as tracking information to monitor the individual 10 and relay the information to the system 168 and/or a computing device as discussed above. Moreover, the systems described herein can be combined with any electronic virtual mapping.

It is noted that the concepts above, while being illustrated as separate applications, can be combined in whole or in part.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation. Unless defined otherwise, all technical and scientific terms used herein have the same meaning as commonly understood by one of ordinary skill in the art to which this invention belongs.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved. Finally, terms of degree such as “substantially”, “about” and “approximately” as used herein mean a reasonable amount of deviation (e.g., a deviation of up to ±0.1%, ±1%, ±5%, or ±10%, as such variations are appropriate) from the specified value such that the end result is not significantly or materially changed.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A method of facilitating direct interaction between a distributed neural network of a brain of an individual and an external device, the method comprising:
generating a plurality of feedback data from the external device where the plurality of feedback data is related to an activity of the external device;
establishing a connection from the external device to a control unit coupled to the individual, where the control unit includes at a first neural implant previously positioned within a first cytoarchitecture region of the distributed neural network of the brain of the individual; and
transmitting the plurality of feedback data to the control unit, such that the control unit energizes the first neural implant to stimulate the first cytoarchitecture region of the brain, which produces an effect in the individual that is specific to the first cytoarchitecture region such that the individual is able to perceive the effect.

2. The method of claim 1, wherein the plurality of feedback data is related to the activity of the external device resulting from actions of the individual.

3. The method of claim 1, wherein transmitting the plurality of feedback data occurs through a network.

4. The method of claim 1, wherein transmitting the plurality of feedback data occurs directly.

5. The method of claim 1, wherein the individual is actively controlling and interacting with the external device during generating the plurality of feedback data.

6. The method of claim 5, wherein the external device is a vehicle.

7. The method of claim 1, further comprising generating a plurality of outfeed data from a neural activity of the first cytoarchitecture region using the first neural implant and transmitting the plurality of outfeed data to the external device.

8. The method of claim 7, wherein transmitting the plurality of outfeed data to the external device comprises transmitting at least one signal command representing the plurality of outfeed data.

9. The method of claim 1, wherein the control unit includes a second neural implant positioned in a second cytoarchitecture region of the distributed neural network of the brain of the individual, and where transmitting the plurality of feedback data to the control unit includes energizing the first neural implant or the second neural implant, to stimulate the first cytoarchitecture region of the brain or the second cytoarchitecture region of the brain.

10. The method of claim 1, wherein the external device comprises an external monitoring server and where the plurality of feedback data assists the individual in decision-making.

11. The method of claim 1, wherein the external device comprises a second control unit or a second electronic device, where the second control unit and the second electronic device are coupled to a second individual.

12. The method of claim 1, wherein the external device comprises a camera system worn by the individual.

13. A method of facilitating direct interaction between a distributed neural network of a brain of an individual and an external device, the method comprising:
generating a plurality of feedback data from the external device;
establishing a connection from the external device to a control unit coupled to the individual, where the control unit includes at a first neural implant previously positioned within a first cytoarchitecture region of the distributed neural network of the brain of the individual; and
transmitting the plurality of feedback data to the individual.

14. The method of claim 13, wherein transmitting the plurality of feedback data to the individual comprises transmitting the plurality of feedback data to the control unit, such that the control unit energizes the first neural implant to stimulate the first cytoarchitecture region of the brain, which produces an effect in the individual specific to the first cytoarchitecture region such that the individual is able to perceive the effect.

15. The method of claim 14, wherein the external device comprises a position tracking system configured to monitor a position of the individual relative to an environment of the individual, wherein generating the plurality of feedback data from the external device comprises information regarding an environmental condition around the individual.

16. The method of claim 13, wherein transmitting the plurality of feedback data to the individual comprises transmitting the plurality of feedback data to an external hardware component which produces an effect perceivable by the individual.

17. The method of claim 16, wherein the external hardware component comprises a camera system worn by the individual.

18. A method of assessing an effect of a medical procedure on a region of interest in a brain of an individual, the method comprising:
positioning at least one endovascular neural monitoring implant within a vessel in the brain adjacent to the region of interest;
engaging the individual to perform one or more tasks that induce a neural activity in the brain; and
measuring the neural activity with the at least one endovascular neural monitoring implant to determine an association between the region of interest and the brain activity for assessing an effect of the medical procedure on the region of interest.

19. The method of claim 18, wherein positioning the at least one endovascular neural monitoring implant within the vessel in the brain adjacent to the region of interest comprises positioning a plurality of endovascular neural monitoring implants within a plurality of vessels in the brain surrounding the region of interest.

20. The method of claim 18, further comprising injecting a substance into the target area prior to measuring the neural activity.

21. The method of claim 20, wherein injecting a substance comprises injecting an anesthetic into an artery targeted for embolization.

22. The method of claim 20, further comprising measuring the neural activity with the at least one endovascular neural monitoring implant after injecting the substance.

23. The method of claim 18, further comprising mapping the one or more tasks to one or more regions of the brain.

24. The method of claim 18, wherein measuring the neural activity further includes measuring the neural activity before and after the procedure.

25. The method of claim 18, wherein the medical procedure includes ablation of a region of tissue.

26.-32. (canceled)
